(19) United States
(12) Patent Application Publication
(10) Pub. No.: US 2015/0366538 A1
(43) Pub. Date: Dec. 24, 2015

(54) HAND HELD ULTRASOUND IMAGING APPARATUS WITH PORTABLE CARRIER CHARGER

(71) Applicant: Analogic Corporation, Peabody, MA (US)

(72) Inventor: Gilbert W. McKenna, Revere, MA (US)

(21) Appl. No.: 14/308,041

(22) Filed: Jun. 18, 2014

(51) Int. Cl. A61B 8/00; H02J 7/00

(52) U.S. Cl. CPC A61B 8/4433; H02J 7/0044; A61B 8/4427; A61B 8/4488; A61B 8/56

(57) ABSTRACT

A system includes a hand-held ultrasound imaging apparatus and a portable charger. The apparatus includes imaging components, an internal power source that powers the imaging components, and at least one electrical contact in electrical communication with the internal power source. The charger includes at least one complementary electrical contact, a charge state monitor in electrical communication with the complementary at least one electrical contact, a charger circuit in electrical communication with the at least one complementary electrical contact and the charge state monitor, and an auxiliary power source in electrical communication with the charger circuit. The charge state monitor, in response to the at least one electrical contact and the at least one complementary electrical contact being in electrical communication, determines a charge state of the internal power source, and the charger circuit charges the internal power source based on the determined charge state and a charge algorithm.

### TECHNICAL FIELD

The following generally relates to a hand-held ultrasound imaging apparatus with a portable carrier charger.

### BACKGROUND

An ultrasound imaging apparatus provides useful information about the interior characteristics of an object under examination. In medical applications, clinicians have used ultrasound imaging apparatuses to examine human subjects in settings such as hospitals, physician's offices, and other locations. Ultrasound imaging apparatuses come in various sizes and configurations, from larger mobile device on rollers, to portable laptop-size devices that can be carried around, to hand-held devices that can be operated with single hand, etc.

FIG. 1 shows an example of a hand-held ultrasound imaging device 100. The hand-held ultrasound imaging device 100 is about the size of a television remote control (e.g., less than twelve inches in length). For example, with the illustrated hand-held ultrasound imaging device 100, on the order of one half to two thirds of the hand-held ultrasound imaging device 100 fits within the palm region of a hand of a user, with a smaller sub-portion(s) 104 extending beyond the palm region, with controls 106 being reachable via the digits of the hand and a display 108 that is not visibly obstructed by the hand.

The smaller package of the device 100 limits the size of the rechargeable battery that can be contained therein. Unfortunately, this may limit the hours of use (e.g., to two hours) before the device 100 needs to be returned to its charger to be recharged, and the total hours of use as the device 100 will need to remain in the charger until a sufficient charge is reached. Furthermore, such devices have been factory sealed, e.g., so that the device 100 can be cleaned without ingress of fluids, and cannot be readily opened, e.g., to replace the rechargeable battery contained therein.

FIG. 2 shows an example base station charger 200 with the device 100 installed therein for charging. The illustrated charger 200 includes at least a power converter 202, such as transformer, that converts alternating current (AC) power to direct current (DC) power, a power cord 204 with a plug 206 configured to plug into a wall receptacle. Generally, such a charger is placed in a charging area and plugged into the wall receptacle. An operator can retrieve the device 100 from the base station charger 200, use the device 100, and return the device 100 to the charger 200 for charging.

The useful life of such the device 100 can be specified by the life of the rechargeable battery contained therein since the consumer cannot replace the internal rechargeable battery. The life of a lithium-ion (Li-ion) rechargeable battery, e.g., is shortened by subjecting the battery to internal stress, such as high charging voltage, high discharge capacity and/or heat. For example, if a Li-ion rechargeable battery is deeply discharged to 80% or 100%, the internal temperature of the battery rises with the amount of electrical current being supplied based on the internal resistance of the battery, and increases heat, reducing the total charge capacity, and hence the life in total charge, discharge cycles.

A balance for battery life is to use a lower charge voltage per cell and then only partially discharge the battery to say 80% of capacity before topping it off (i.e., replacing the 20%). Unfortunately, this balance is not well-suited to practical applications. For example, it may not be practical when imaging patients to return the device 100 to the charger 200 after every use or partial discharge. This may be especially true when the time between uses is the commuting time between patients. Moreover, the device 100 may not be able to be returned to the charger 200 until the end of a shift or the day.

### SUMMARY

Aspects of the application address the above matters, and others.

In one aspect, a system includes an ultrasound imaging apparatus and a portable charger. The apparatus includes imaging components, an internal power source that powers the imaging components, and at least one electrical contact in electrical communication with the internal power source. The charger includes at least one complementary electrical contact, a charge state monitor in electrical communication with the complementary at least one electrical contact, a charger circuit in electrical communication with the at least one complementary electrical contact and the charge state monitor, and an auxiliary power source in electrical communication with the charger circuit. The charge state monitor, in response to the at least one electrical contact and the at least one complementary electrical contact being in electrical communication, determines a charge state of the internal power source, and the charger circuit charges the internal power source based on the determined charge state and a charge algorithm. In another aspect, a method includes sensing an electrical contact of a hand-held ultrasound imaging apparatus is in electrical communication with a complementary electrical contact of the ultrasound imaging apparatus portable charger, sensing a charge capacity of an internal power source of the hand-held ultrasound imaging apparatus with the ultrasound imaging apparatus portable charger, and charging the internal power source of the hand-held ultrasound imaging apparatus with the ultrasound imaging apparatus portable charger based on the sensed charge capacity and a charge algorithm.

In another aspect, a hand-held electronic device carrying case includes a receiving region configured to receive a hand-held electronic device. The hand-held electronic device carrying case further includes an electrical contact disposed at least partially in the receiving region. The hand-held electronic device carrying case further includes a charge state monitor that monitors a charge state of the electronic device installed in the receiving region and in electrical communication with the electrical contact. The hand-held electronic device carrying case further includes a charger circuit that charges the electronic device installed in the receiving region through the electrical contact based on the charge state and a charge algorithm, which tops a charge of the hand-held electronic device.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 illustrates an example prior art hand-held ultrasound imaging apparatus;

FIG. 2 illustrates the example prior art hand-held ultrasound imaging apparatus of FIG. 1 installed in an example prior art base charging unit located at a charge station;

FIG. 3 schematically illustrates an example system including a hand-held ultrasound imaging apparatus and a hand-held ultrasound imaging apparatus portable charger unit;

FIG. 4 schematically illustrates an example of the hand-held ultrasound imaging apparatus charger unit of FIG. 3 in connection with the hand-held ultrasound imaging apparatus of FIG. 3;

FIG. 5 schematically illustrates another example of the hand-held ultrasound imaging apparatus portable charger unit of FIG. 3 in connection with a charger for the portable charger;

FIG. 6 schematically illustrates another example the hand-held ultrasound imaging apparatus portable charger unit of FIG. 3 with an internal charger;

FIG. 7 schematically illustrates an example showing connection between the hand-held ultrasound imaging apparatus, the hand-held ultrasound imaging apparatus portable charger, and a base charger;

FIG. 8 schematically illustrates an example with the hand-held ultrasound imaging apparatus is installed in the hand-held ultrasound imaging apparatus portable charger, which is installed in the base charger;

FIG. 9 schematically illustrates another example showing connections between the hand-held ultrasound imaging apparatus, hand-held ultrasound imaging apparatus the portable charger, and a base charger;

FIG. 10 schematically illustrates an example showing connections between the hand-held ultrasound imaging apparatus, the hand-held ultrasound imaging apparatus portable charger, a base charger, and a hand-held ultrasound imaging apparatus portable charger base charger;

FIG. 11 illustrates a top-down perspective view of the hand-held ultrasound imaging apparatus with charging electrical contacts disposed on the bottom;

FIG. 12 illustrates a bottom-up perspective view of the hand-held ultrasound imaging apparatus with charging electrical contacts disposed on the back;

FIG. 13 illustrates a top-down perspective view of the hand-held ultrasound imaging apparatus with charging electrical contacts disposed on the front;

FIG. 14 illustrates a top-down perspective view of the hand-held ultrasound imaging apparatus with charging electrical contacts disposed on the side;

FIG. 15 illustrates an example of the hand-held ultrasound imaging apparatus portable charger without the hand-held ultrasound imaging apparatus;

FIG. 16 illustrates an example of the hand-held ultrasound imaging apparatus portable charger with the hand-held ultrasound imaging apparatus installed therein;

FIG. 17 illustrates an example utility tray carrying the portable charger with the hand-held ultrasound imaging apparatus installed therein; and

FIG. 18 illustrates an example method.

### DETAILED DESCRIPTION

FIG. 3 schematically illustrates a system 300 that includes an ultrasound imaging apparatus (UIA) 302 and an ultrasound imaging apparatus (UIA) charger 304. The illustrated ultrasound imaging apparatus 302 is a hand-held ultrasound imaging (HHUI) device, which includes a single enclosure housing 306, which houses and/or physically supports the components therein.

An example of a HHUI device is described in U.S. Pat. No. 7,699,776 B2, filed on Sep. 7, 2004, and entitled “Intuitive Ultrasonic Imaging System and Related Method thereof,” which is incorporated herein by reference in its entirety. Another example is described in Fuller et al., “Real Time Imaging with the Sonic Window: A pocket-Sized, C-scan, Medical Ultrasound Device,” 2009 IEEE International Ultrasonics Symposium Proceedings, pp. 196-199, 2009.

The ultrasound imaging apparatus 302 includes a transducer array 308, such as a one-dimensional (1D) or two-dimensional (2D) transducer array, with one or more transducer elements 310 arranged in a linear, curved, circular, or other manner. For example, the transducer array 308 may include a thirty-two by thirty-two, a sixty-four by sixty-four, . . . , a two hundred and fifty-six by two hundred and fifty-six, or other array of the transducer elements 310, including a non-square array such as rectangular, circular, and/or other array.

The illustrated transducer array 308 is configured to acquire data for C-plane/scan, A-mode, B-mode, etc. acquisitions, individually and in combination with color flow, Doppler flow, elastography, contrast harmonic, and/or other information. C-plane/scan imaging, generally, can include displaying images parallel to the skids surface, giving the impression of viewing the tissue of interest with the perspective of a clear “window” through the skin.

An example of C-plane/scan imaging is described in U.S. Pat. No. 7,402,136 B2, filed on Jul. 14, 2005, and entitled “Efficient Ultrasound System for Two-Dimensional C-Scan Imaging and Related Method Thereof,” which is incorporated herein by reference in its entirety. Another example of C-plane/scan imaging is described in Fuller et al., “Real Time Imaging with the Sonic Window: A pocket-Sized, C-scan, Medical Ultrasound Device,” 2009 IEEE International Ultrasonics Symposium Proceedings, pp. 196-199, 2009. Other approaches to C-plane/scan imaging are also contemplated herein.

The ultrasound imaging apparatus 302 further includes transmit circuitry 312, receive circuitry 314, and a switch 316 that switches between the transmit and the receive circuitry 312 and 314. Generally, the transmit circuitry 312 controls excitation of the transducer elements 310, which allows for steering and/or focusing the transmitted beam from an origin along the array and at a predetermined angle, and the receive circuitry 314 routes echo signals received by the transducer elements 310.

The ultrasound imaging apparatus 302 further includes an echo processor 318 that processes received echo signals. Such processing may include beamforming (e.g., delay and sum, etc.) and/or other processing to, e.g., lower speckle, improve specular reflector delineation, filter the echo signals (via FIR and/or IIR), etc.

The ultrasound imaging apparatus 302 further includes a controller 320 that controls the transmit circuitry 312, the receive circuitry 314, and the echo processor 318. Such control may include controlling the frame rate, transmit angles, energies and/or frequencies, transmit and/or receive delays, processing of echo signals, the imaging mode, etc.

The ultrasound imaging apparatus 302 further includes a scan converter 322, which coverts processed echo signals and generates data for display, a display 324, which displays the scan converted data, and a user interface 326, which includes input controls and/or output displays for interacting with the system 300. At least a sub-portion of the display 324 and/or the user interface 326 may be disposed within, integrated with and/or part of the housing 306.

The ultrasound imaging apparatus 302 further includes an internal power source 328, which includes one or more of a rechargeable battery (e.g., Li-ion, nickel-cadmium (NiCad), nickel-metal hydride (NiMH), lead acid, etc.), a super capacitor and/or other power storage device, which supplies power to one or more of the electrical components 308-326. The internal power source 328 is disposed within or fully internal to the housing 306 so as not to be readily replaceable by the user.

The ultrasound imaging apparatus 302 further includes at least one electrically conductive contact 330, which is disposed at least partially outside of the housing 306. An electrically conductive path 332, such as a trace, a wire, etc., electrically connects the internal power source 328 and the at least one electrically conductive contact 330.

The UIA portable charger 304 includes at least one complementary electrical contact 334. The at least one complementary electrical contact 334 is disposed at least partially exposed such that it can be physically contacted by from the outside of the UIA portable charger 304. The at least one complementary electrical contact 334 is complementary to the contact 330 in that it physically contacts the at least one electrically conductive contact 330 when the ultrasound imaging apparatus 302 is installed in the UIA portable charger 304.

The UIA portable charger 304 is a portable device configured to receive at least the at least one electrically conductive contact 330 of the ultrasound imaging apparatus 302. In one non-limiting instance, the UIA portable charger 304 serves as a carrying case device for the ultrasound imaging apparatus 302. In this configuration, the UIA portable charger 304 may cover a substantial portion of (e.g., 30%, 50%, 75%, etc.) or all of the ultrasound imaging apparatus 302 and protect the ultrasound imaging apparatus 302 from the environment, while recharging the internal power source 328.

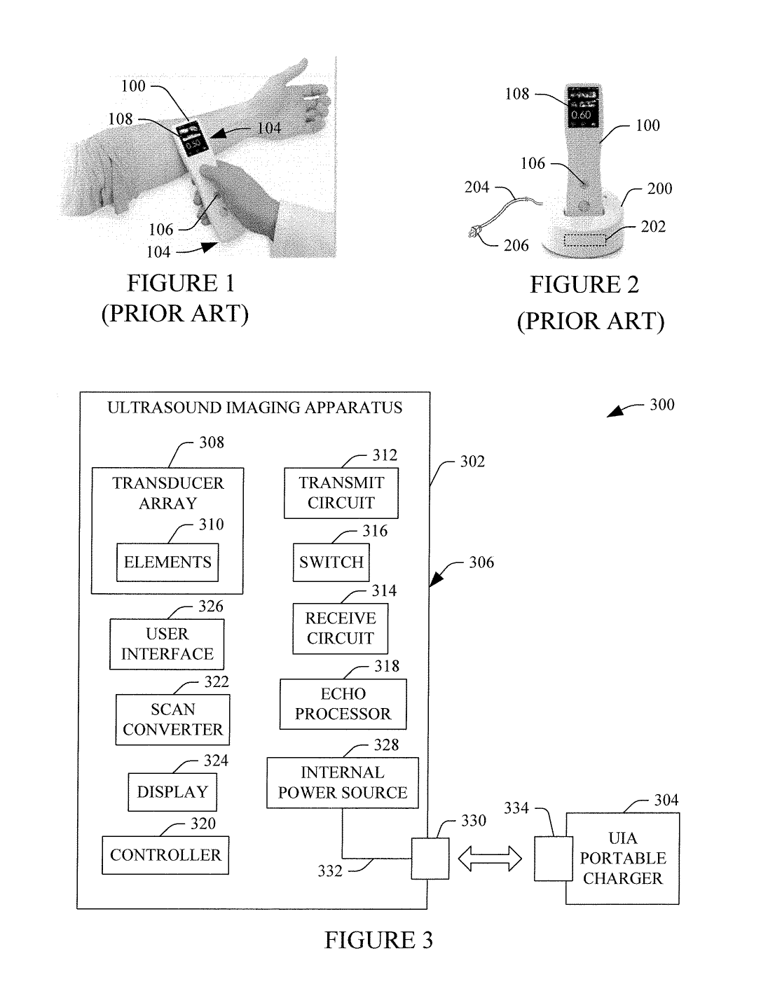

Unlike the charger 200 of FIG. 2, the UIA portable charger 304 does not include a transformer or a power cord. As such, the footprint of the UIA portable charger 304 is smaller than the footprint of the charger 200, and the UIA portable charger 304 is lighter than (weighs less) the charger 200. In one instance, the UIA portable charger 304 has a geometry and weight well-suited to carry along with the ultrasound imaging apparatus 302 from patient to patient in a utility tray, a pocket, an equipment cart, etc. with the rest of the items needed for drawing blood, starting an IV, etc.

The UIA portable charger 304 is configured to top off the charge of the internal power source 328, for example, when the ultrasound imaging apparatus 302 is installed in the UIA portable charger 304 so that a current capacity is always above a threshold amount. By way of example, where the threshold amount is 80%, the UIA portable charger 304 charges the installed the internal power source 328 in response to a current capacity of the internal power source 328 being below 80%. In other instance, the percentage can be in a range from 50% to 99%.

As such, the life of the internal power source 328 and hence the life of the ultrasound imaging apparatus 302 can be extended, relative to not using the UIA portable charger 304. For example, discharging a Li-ion battery to 90 to 100% of capacity may reduce the useful life to about one year, or 300-500 discharge cycles, and where the voltage per cell is 4.3 volts, this further reduces the total discharge cycles to approximately 150 to 250, and heating from charging further reduces the total discharge cycles. The topping off of the UIA portable charger 304 can extend the life of such a battery 3 to 10 years.

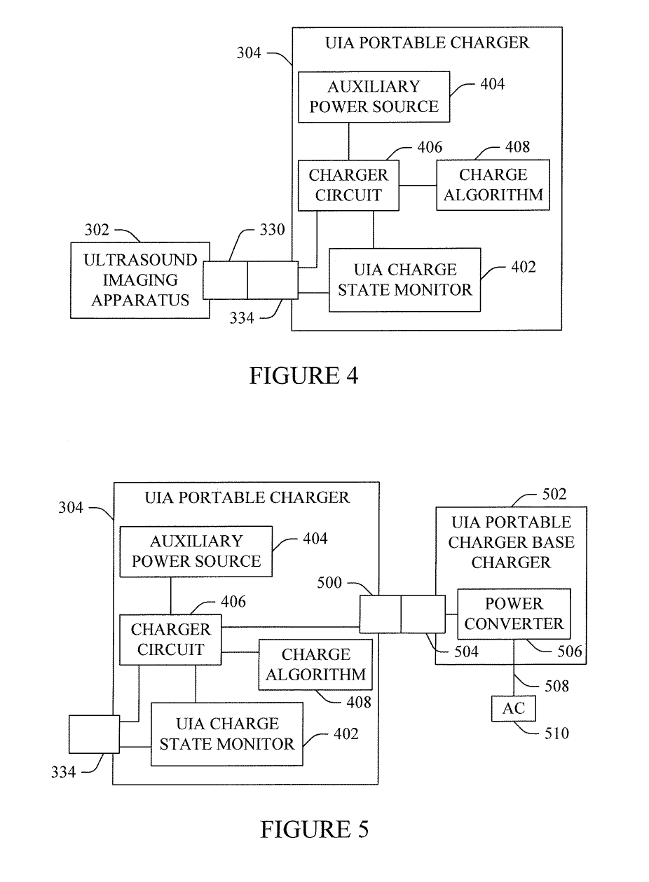

Turning to FIG. 4, a non-limiting example of the UIA portable charger 304 is illustrated.

In this example, the UIA portable charger 304 includes an ultrasound imaging apparatus (UIA) charge monitor 402. The UIA charge monitor 402 monitors a charge of the internal power source 328 of the ultrasound imaging apparatus 302 in response to an electrical connection between the at least one electrical contact 330 of the ultrasound imaging apparatus 302 and the at least one complementary electrical contact 334 of the UIA portable charger 304.

The UIA portable charger 304 further includes an auxiliary power source 404, a charger circuit 406, and at least one charge algorithm 408. The auxiliary power source 404 can be a primary cell or non-rechargeable battery (e.g., alkaline, zinc-carbon, etc.), a secondary cell or rechargeable battery (e.g., Li-ion, NiCad, NiMH, lead acid, etc.), a super capacitor, or other power storage device. The auxiliary power source 404 is disposed such that it is readily replaceable by the user. The charger circuit 406 charges the internal power source 328, through the electrical contacts 330 and 334, with the auxiliary power source 404 based on the monitored charge.

For charging, the charger circuit 406 converts electrical energy from auxiliary power source 404 to a voltage and/or a current level suitable for charging the internal power source 328. The charging is based on a charge algorithm of interest of the at least one charge algorithm 408. An example charge algorithm supplies charge from the auxiliary power source 404 to the internal power source 328 in response to the monitored charge falling below a predetermined threshold charge (e.g., 99.9%, 90%, 85%, 78%, 50%, or other threshold), the electrical contact 330 and 334 being in physical contact.

In another example, a charge algorithm supplies charge from the auxiliary power source 404 to the internal power source 328 simply by placing the at least one electrical contact 330 and the at least one complementary electrical contact 334 come in physical contact, if the UIA portable charger 304 is activated to charge. Charge mode may always be active or activated via an on/off mechanism (e.g., a switch, a button, etc.). Other algorithms are also contemplated herein.

Turning to FIG. 5, another example of the UIA portable charger 304 is illustrated. In this embodiment, the UIA portable charger 304 is substantially similar to that described in connection with FIG. 4 and additionally includes a second electrical contact 500. The second electrical contact 500 receives charge that is used by the charger circuit 406 to charge a rechargeable auxiliary power source 404.

A UIA portable charger base charger 502 includes a power converter 506. The power converter 506 converts AC power, for example, from an AC receptacle, to DC, and conveys the DC power to the charger circuit 406. The UIA portable charger base charger 502 includes complementary electrical contact 504. An electrical connector 508, such as a power cord, a line cord, a mains cable, etc. includes a plug that removeably plugs into the mains electricity supply via a wall socket, an extension cord, etc.

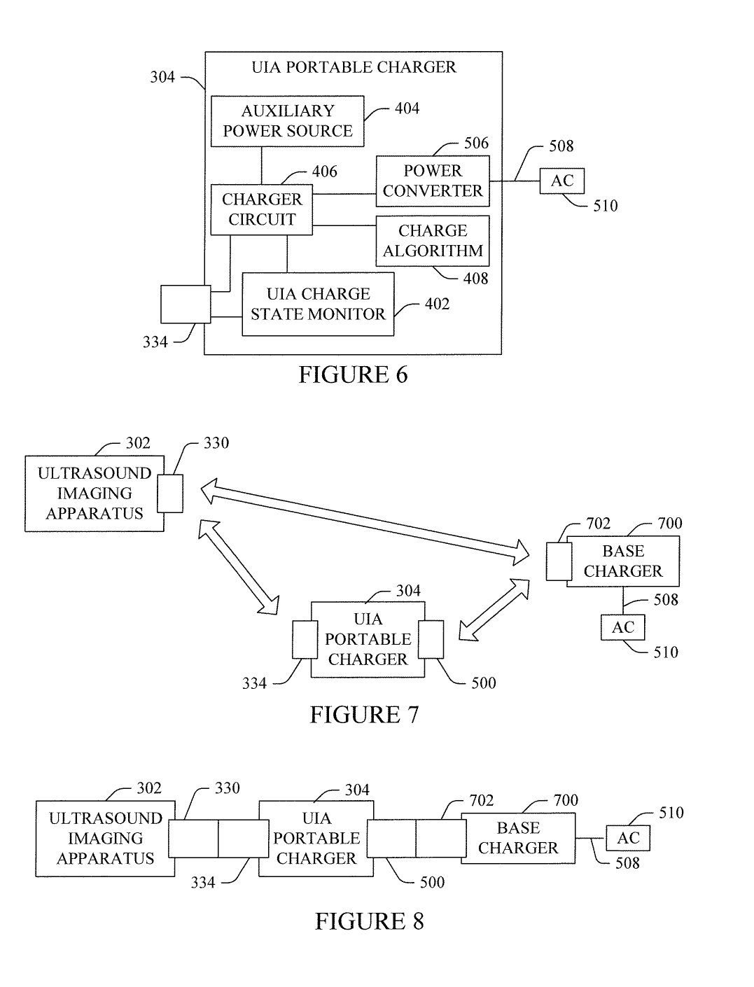

FIG. 6 shows a variation of FIG. 5 in which the UIA portable charger 304 includes and the power converter 506. Likewise, the power converter 506 receives and converts AC power to DC and conveys the DC power to the charger circuit 406.

FIG. 7 shows an embodiment in which a same base charger 700 can be used to alternatively charge the internal rechargeable battery 328 of the ultrasound imaging apparatus 302 and the auxiliary battery 404 of the UIA portable charger 304. The base charger 700, in this example, is substantially similar to or the same as the UIA portable charger 304. In a variation, the chargers 700 and 304 are different.

As shown in FIG. 8, in one embodiment, the ultrasound imaging apparatus 302 can be installed in the UIA portable charger 304 when the UIA portable charger 304 is installed in the base charger 700. In one instance, the base charger 700 can concurrently charge both the internal power source 328 and the auxiliary power source 404. In another instance, the base charger 700 can charge only one of the internal power source 328 and the auxiliary power source 404 at any given time, or only the auxiliary power source 404 and not the internal power source 328.

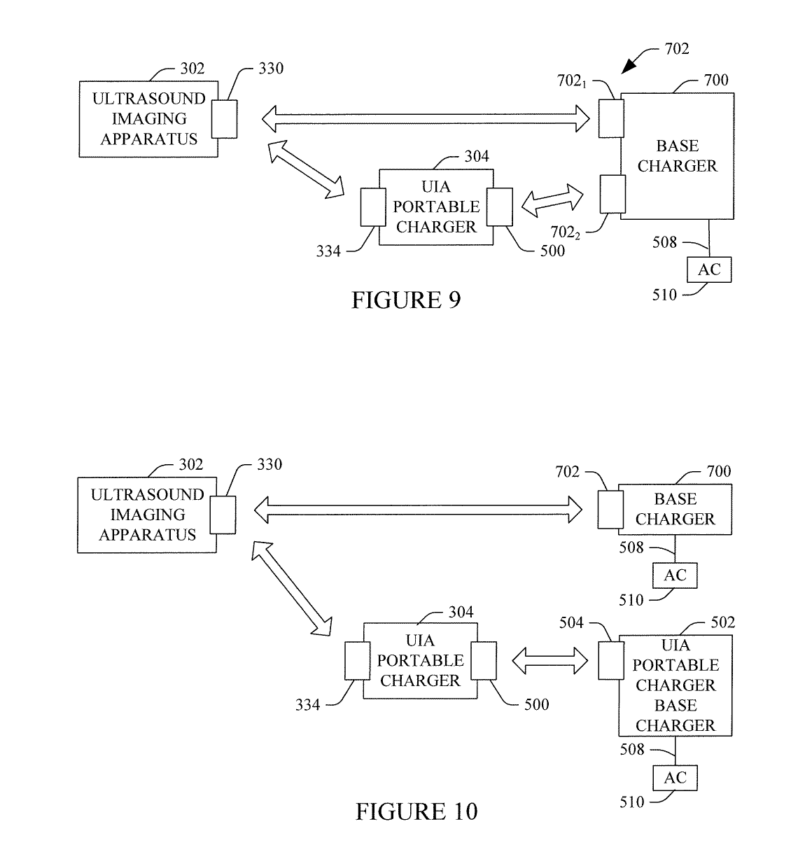

FIG. 9 shows an embodiment in which the electrical contact 702 of the base charger 700 includes a first set of electrical contact 702₁ and a second set of electrical contact 702₁. The first set of electrical contact 702₁ is complementary to the electrical contact 330 of the ultrasound imaging apparatus 302, and the second set of electrical contact 702₂ is complementary to the electrical contact 500 of the UIA portable charger 304. Similar to FIG. 8, the base charger 700 concurrently and/or individually charges the internal power source 328 and the auxiliary power source 404.

FIG. 10 shows an embodiment in which the UIA portable charger base charger 502 charges the auxiliary power source 404 as described in FIG. 5 and/or otherwise, and the base charger 700 charges the internal power source 328 as described in FIG. 7 and/or otherwise. In this example, the UIA portable charger 304 charges the internal power source 328 as described in FIG. 4 and/or otherwise.

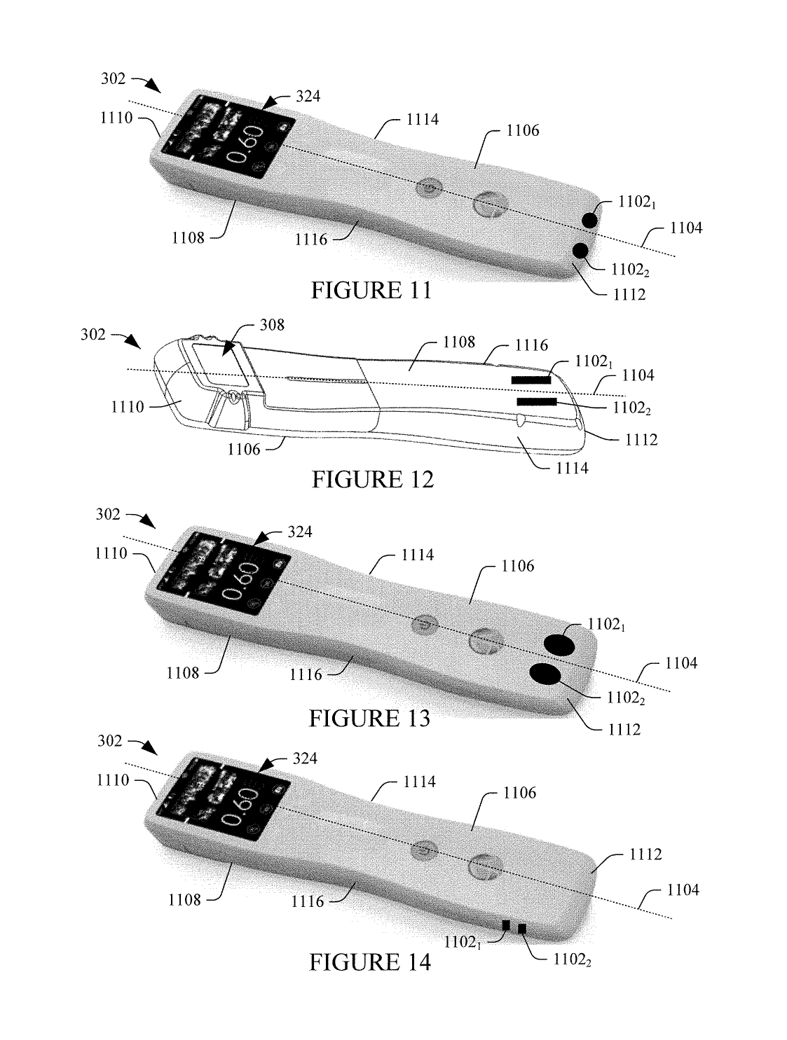

FIGS. 11-14 show various example locations of the at least one electrical contact 330. In these examples, the at least one electrical contact 330 at least includes contacts 1102₁ and 1102₂. FIGS. 11, 12 and 14 show a top down perspective view from a front side, and FIG. 13 shows a bottom up perspective view from a back side.

The ultrasound imaging apparatus 302 includes a long axis 1104, a first or display and control side 1106 in which the display 324 and the user interface 326 are accessed, a second or imaging side 1108 in through which ultrasounds signals are transmitted and received, a third or top side 1110 adjacent the transducer array 308, a fourth or bottom side 1112 located on an opposing end of the long axis 1104 from the top side 1110, and a fifth side 1114 and a sixth side 1116, each of which extend between the first, second, third and fourth sides, on opposing sides of the long axis 1104.

In FIG. 11, the contacts 1102₁ and 1102₂ are generally circular in shape and are located on the fourth or bottom side 1112. In FIG. 12, the contacts 1102₁ and 1102₂ are generally rectangular in shape and are located on the second or imaging side 1108. In FIG. 13, the contacts 1102₁ and 1102₂ are generally elliptical in shape and are located on the first or display and control side 1106. In FIG. 14, the contacts 1102₁ and 1102₂ are generally square in shape and are located on the fifth side 1114.

Other locations for the contacts 1102₁ and 1102₂ of the ultrasound imaging apparatus 302 are also contemplated herein. Furthermore, the relative sizes of the contacts 1102₁ and 1102₂ are provided for explanatory purposes and are not limiting. Moreover, the shape of the ultrasound imaging apparatus 302 is provided for explanatory purposes and are not limiting.

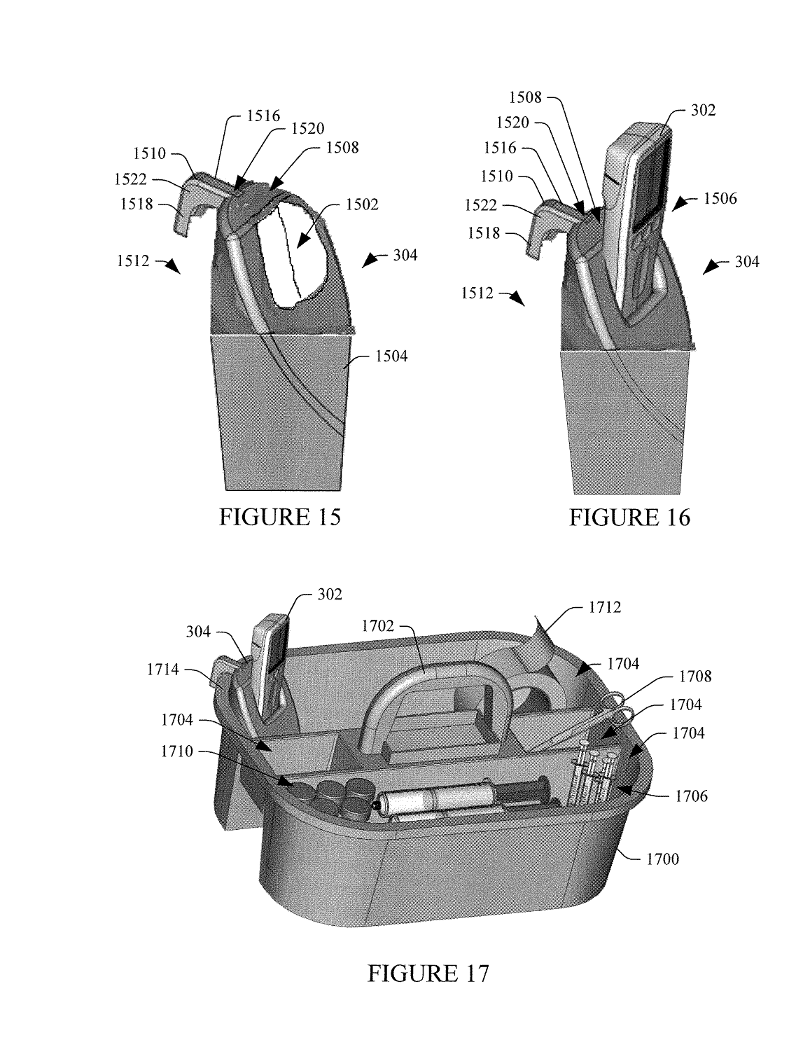

FIGS. 15 and 16 illustrate an example of the UIA portable charger 304. FIG. 15 shows the UIA portable charger 304 without the ultrasound imaging apparatus 302 installed therein, and FIG. 16 shows the UIA portable charger 304 with the ultrasound imaging apparatus 302 installed therein. From FIGS. 15 and 16, in one instance, the UIA portable charger 304 includes a material free recess 1502 that geometrically configured to receive the ultrasound imaging apparatus 302.

In this embodiment, a lower sub-portion of the material free recess 1502 is entirely surrounded by an outer physical shell 1504, and an upper sub-portion of the material free recess 1502 is only partially, and not entirely, surrounded by the outer physical shell 1504. The outer physical shell 1504 shields the sub-portion of ultrasound imaging apparatus 302 in the recess 1502 from debris. Furthermore, a depth of the material free recess 1502 is such that only a sub-portion 1506 of the ultrasound imaging apparatus 302 protrudes out of the material free recess 1502, extending beyond a top 1508 of the UIA portable charger 304.

The illustrated UIA portable charger 304 further includes a member 1510 that protrudes out of a back 1512 of the UIA portable charger 304. The illustrated UIA member 1510 includes a first sub-portion 1516 that extends generally perpendicular from the back 1512, and a second sub-portion 1518 that extends generally parallel to the back 1512 and transverse to the first sub-portion 1516. The first sub-portion 1516 is attached to the back 1512 at a first region 1520, and the second sub-portion 1518 is affixed to the first sub-portion at a second region 1522 of the first sub-portion 1516, which is opposite the first region 1520. In one instance, the member 1510 forms a hook, bracket, or the like.

FIG. 17 shows an example of the UIA portable charger 304 of FIGS. 15 and 16 in which the UIA portable charger 304 is installed in a utility tray 1700. The illustrated utility tray 1700 includes a handle 1702 and compartments 1704 for holding items. In the context of phlebotomist, the utility tray 1700 can be used to carry items such as syringes 1706, scissors 1708, vials 1710, tape 1712, etc. In FIG. 17, the UIA portable charger 304 is shown secured in a compartment 1704, with the member 1510 placed over a wall 1714, which facilitates securing the UIA portable charger 304 in the utility tray 1700.

The user can carry the utility tray 1700 from patient to patient, removing the ultrasound imaging apparatus 302 from the UIA portable charger 304 to use the ultrasound imaging apparatus 302 to facilitate, for example, drawings blood, inserting an IV line, etc. Once fished, the user can clean and return the ultrasound imaging apparatus 302 back to the UIA portable charger 304, where the charge of the internal power source 328 of the ultrasound imaging apparatus 302 can be topped off by the auxiliary power source 404 of the UIA portable charger 304 as described herein and/or otherwise.

Although the above has been described in connection with an example of the ultrasound imaging apparatus 302, it is to be understood that the embodiments disclosed herein, generally, relate to any hand-held electronic device powered with a rechargeable battery. For example, the portable charger 304 can be used with cell phones, smart phones, remote controls, hand-held gaming system, etc.

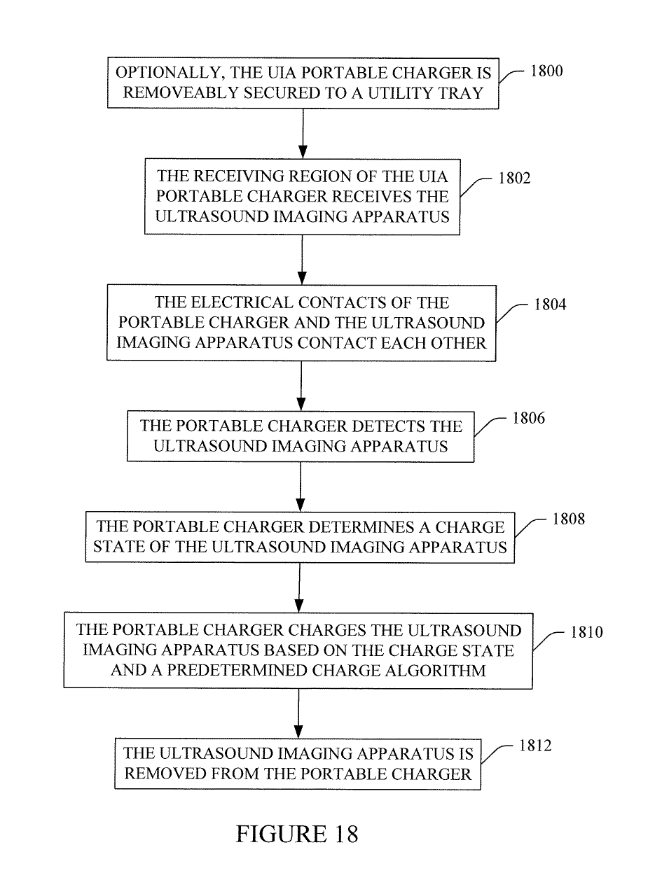

FIG. 18 illustrates an example method.

It is to be understood that the following acts are provided for explanatory purposes and are not limiting. As such, one or more of the acts may be omitted, one or more acts may be added, one or more acts may occur in a different order (including simultaneously with another act), etc.

At 1800, optionally, the UIA portable charger 304 is removeably secured to the utility tray 1700.

At 1802, the receiving region 1502 of the UIA portable charger 304 receives the ultrasound imaging apparatus 302.

At 1804, the at least one electrical contact of the ultrasound imaging apparatus 302 electrically contacts the at least one complementary electrical contact of the UIA portable charger 304.

At 1806, the UIA portable charger 304 detects the received ultrasound imaging apparatus 302.

At 1808, the UIA charge state monitor 402 of the UIA portable charger 304 determines a charge state of the internal power source 328 of the ultrasound imaging apparatus 302.

At 1810, the charger circuit 406 of the UIA portable charger 304, based on the determined charge state of the internal power source 328 of the ultrasound imaging apparatus 302 and a predetermined charge algorithm, tops off the charge of the internal power source 328 of the ultrasound imaging apparatus 302.

At 1812, the ultrasound imaging apparatus 302 is removed from the UIA portable charger 304.

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. A system, comprising:
a hand-held ultrasound imaging apparatus, including: imaging components; an internal power source that supplies power that powers at least one of the imaging components; and at least one electrical contact in electrical communication with the internal power source; and
an ultrasound imaging apparatus portable charger, including: at least one complementary electrical contact; a charge state monitor in electrical communication with the complementary at least one electrical contact; a charger circuit in electrical communication with the at least one complementary electrical contact and the charge state monitor; and an internal auxiliary power source in electrical communication with the charger circuit,
wherein the charge state monitor, in response to the at least one electrical contact and the at least one complementary electrical contact being in electrical communication, determines a charge state of the internal power source, and the charger circuit charges the internal power source based on the determined charge state and a predetermined charge algorithm.

2. The system of claim 1, wherein the charge algorithm causes the charger circuit to charge the internal power source in response to the determined charge state of the internal power source being below a predetermined threshold percentage of a full charge.

3. The system of claim 2, wherein the predetermined threshold percentage of the full charge is eighty percent.

4. The system of claim 1, wherein the charge algorithm causes the charger circuit to charge the internal power source in response to the determined charge state being less than a full charge.

5. The system of claim 1, wherein the internal power source includes a rechargeable storage element.

6. The system of claim 5, wherein the rechargeable storage element is a rechargeable battery or a super capacitor.

7. The system of claim 5, wherein the internal power source is non-replaceable.

8. The system of claim 1, wherein the auxiliary power source includes one or more of a rechargeable battery, a non-rechargeable battery, or a super capacitor.

9. The system of claim 8, wherein the auxiliary power source includes one or more of a lithium-ion battery, a nickel-cadmium battery, a nickel-metal hydride battery, a lead acid battery, an alkaline, or a zinc-carbon battery.

10. The system of claim 1, wherein the portable charger does not include a power converter that converts alternating current power into direct current power.

11. The system of claim 1, wherein the portable charger is part of an hand-held ultrasound imaging apparatus carrying case.

12. The system of claim 11, wherein the portable charger includes a bracket configured to removeably attach to a portable utility tray.

13. The system of claim 1, wherein the portable charger entirely surrounds at least a sub-portion of the hand-held ultrasound imaging apparatus.

14. The system of claim 13, wherein the portable charger shields the at least the sub-portion of the hand-held ultrasound imaging apparatus from debris.

15. A method, comprising:
sensing an electrical contact of a hand-held ultrasound imaging apparatus is in electrical communication with a complementary electrical contact of the ultrasound imaging apparatus portable charger;
sensing a charge capacity of an internal power source of the hand-held ultrasound imaging apparatus with the ultrasound imaging apparatus portable charger; and
charging the internal power source of the hand-held ultrasound imaging apparatus with the ultrasound imaging apparatus portable charger based on the sensed charge capacity and a charge algorithm.

16. The method of claim 15, further comprising:
charging the internal power source of the hand-held ultrasound imaging apparatus with the ultrasound imaging apparatus portable charger in response to the charge capacity being less than a predetermined percent of full capacity.

17. The method of claim 15, wherein the internal power source includes a rechargeable storage element that is not replaceable.

18. The method of claim 15, wherein the ultrasound imaging apparatus portable charger does not convert alternating current to direct current.

19. The method of claim 15, wherein the ultrasound imaging apparatus portable charger is a portable ultrasound imaging apparatus protective carrying case.

20. A hand-held electronic device carrying case, comprising:
a receiving region configured to receive a hand-held electronic device;
an electrical contact disposed at least partially in the receiving region;
a charge state monitor that monitors a charge state of the electronic device installed in the receiving region and in electrical communication with the electrical contact; and
a charger circuit that charges the electronic device installed in the receiving region through the electrical contact based on the charge state and a charge algorithm, which tops a charge of the hand-held electronic device.
